(19) United States
(12) Patent Application Publication
(10) Pub. No.: US 2019/0177506 A1
(43) Pub. Date: Jun. 13, 2019

(54) USE OF EPOXIDIZED FATTY ACID ESTER TO PLASTICIZE NITROCELLULOSE

(71) Applicant: PolyOne Corporation, Avon Lake, OH (US)

(72) Inventors: Alan BARCON, Valley View, OH (US); Stephen D. HORTON, Avon, OH (US)

(73) Assignee: PolyOne Corporation, Avon Lake, OH (US)

(21) Appl. No.: 16/308,010

(22) PCT Filed: Jun. 7, 2017
(86) PCT No.: PCT/US2017/036415

§ 371 Date: Dec. 7, 2018

Related U.S. Application Data
(60) Provisional application No. 62/347,455, filed on Jun. 8, 2016.

(51) Int. Cl. C08K 5/1515; C08L 1/18; A61K 8/73; A61K 8/92; A61Q 3/02

(52) U.S. Cl. CPC C08K 5/1515; C08L 1/18; A61Q 3/02; A61K 8/922; A61K 8/731

(57) ABSTRACT

Nitrocellulose is plasticized with epoxidized fatty acid esters, such as epoxidized alkyl soyates. Nail varnish can be made from the plasticized nitrocellulose.

### CLAIM OF PRIORITY

This application claims priority from U.S. Provisional Patent Application Ser. No. 62/347,455 bearing Attorney Docket Number 1201620 and filed on Jun. 8, 2016, which is incorporated by reference.

### FIELD OF THE INVENTION

This invention relates to the use of epoxidized ester from a fatty acid, such as epoxidized alkyl soyates, as plasticizers for nitrocellulose, a film-forming material.

### BACKGROUND OF THE INVENTION

Plasticizers from petroleum feedstocks and other synthetic sources have been dominant in industry since the mid-Twentieth Century following the polymerization of vinyl chloride and a need to make that polyvinyl chloride flexible. Phthalate plasticizers have been most prevalent.

Plasticizers from biological, renewable sources have been explored in recent years because of concerns about availability of petroleum feedstocks, cost, and asserted health concerns.

U.S. Pat. No. 6,797,753 (Benecke et al.) teaches the manufacture of a number of epoxidized esters from fatty acids, including epoxidized propylene glycol disoyate and epoxidized methyl soyate (EMS).

Such bio-plasticizers have been proposed for use with cellulose alkanoates, such as in PCT Patent Publication No. WO 2011/046736 (Zhu et al.).

U.S. Pat. No. 5,676,935 (Mellul et al.) have proposed making nail polish using nitrocellulose and epoxidized soybean oil (ESO).

However, EMS differs significantly from ESO. ESO is simply an epoxidized naturally occurring triglycercide, whereas EMS, while based on biological feedstocks, is a synthetically derived and epoxidized mono-ester having a significantly different solubility parameter which makes it more effective and efficient at plasticizing and/or coalescing polar polymers such as nitrocellulose.

### SUMMARY OF THE INVENTION

What the art needs is a bio-derived plasticizer, namely an epoxidized ester from a fatty acid, for use with nitrocellulose or other film forming polymers useful in cosmetics, particularly nail polish.

The present invention solves the problem in the art by using an epoxidized fatty acid ester, such as epoxidized alkyl soyate, and more particularly epoxidized methyl soyate (EMS), to plasticize nitrocellulose (or other film-forming polymer resins conventionally used in cosmetics) at ambient temperature or at elevated temperature, depending on the particular ester and the particular film-forming polymer.

The interaction of plasticizers with the polymers or other chemicals to be plasticized should be understood by those having ordinary skill in the art. But for completeness, the theory most prevalent concerning plasticizers is that the continuum of concentric circles around a nominal polymer resin from a highly solvating plasticizer in the closest ring to a mere external lubricant at the outermost ring needs to be identified for each polymer resin, because the value of the plasticizer to associate with a polymer resin particle and render it less stiff exists between those extreme rings. The highly solvating plasticizer will dissolve the polymer resin. The external lubricant will not penetrate the particle of polymer resin. Neither is satisfactory for a plasticized polymer resin.

Moreover, whether a plasticizer is to be used alone or in combination with other plasticizers is quite dependent on whether the candidate plasticizer is to be the primary plasticizer or a secondary plasticizer.

There are several different interpretations about what constitutes a primary plasticizer. For purposes of this invention, a “primary” plasticizer will denote both the plasticizer with the largest mass component in the plasticized polymer composite and also the plasticizer with the most significant plasticizing effect on the polymer resin(s).

Within the concentric circles of plasticization, for any given polymer or polymer family, only experimentation will yield the data necessary to understand which plasticizers will work with which polymer resins. Often, the actual plasticization results are unpredictable and surprising because establishment of a solubility parameter scale, such as the Hillebrand Solubility Parameter, is an exercise in hindsight reconstruction of what actually exists, by application of a theory.

The technology is further complicated by different performances at different temperatures. Certainly, ambient temperature performance is vital for use, but also elevated temperature for plasticization is also vital for efficient manufacturing.

Unexpectedly, it has been found that epoxidized fatty acid esters such as alkyl monosoyates are more effective as plasticizers than epoxidized vegetable oils, because they have a lower molecular weight and are more polar, thus exhibiting a higher solubility parameter closer to that of nitrocellulose.

Therefore, one aspect of the present invention is a plasticized composite of bio-derived materials, comprising (a) a nitrocellulose and (b) an epoxidized fatty acid ester plasticizer mixed in an amount to sufficiently to reduce flexural modulus of the nitrocellulose when in a film upon a human fingernail or toenail.

Another aspect of the present invention is a method of using an epoxidized alkyl soyate as a plasticizer for nitrocellulose comprising the step of mixing the soyate with the nitrocellulose at a temperature sufficient to form a plasticized composite described above.

Another aspect of the present invention is an article of the plasticized composite described above.

Other features and advantages of the invention will be explained below.

### EMBODIMENTS OF THE INVENTION

Epoxidized Ester from a Fatty Acid

Any plasticizer containing fatty acids derived from vegetable oils and which have been substantially fully esterified with an alcohol (monool or polyol) and having unsaturated bonds that are substantially fully epoxidized is a candidate for use as a plasticizer herein. Moreover, it is helpful if the fatty acids are added substantially randomly to one or more hydroxyl sites on the alcohol.

U.S. Pat. No. 6,797,753 (Benecke et al.), the disclosure of which is incorporated herein by reference, teaches the value of these epoxidized esters from fatty acids, including manufacture of a number of epoxidized esters from fatty acids, including epoxidized propylene glycol disoyate and epoxidized methyl soyate (EMS).

Benecke et al. teach the use of any vegetable or plant fatty acid glyceride that is significantly unsaturated. “Significantly unsaturated” means that the vegetable oil typically has more than about 80% unsaturated fatty adds. Most preferably the unsaturation should be about 84% or higher. Typically, the oil has an iodine value, which is a measurement of the amount of double bonds in the fatty acids of the oil that is about 100 and higher.

Iodine Value (IV) is a measure of unsaturation and a good indicator of the extent of possible epoxidation.

Examples of acceptable oils as sources for fatty acid derivatives and their respective IVs include: soybean oil (IV about 120-143), canola oil (IV about 100-115), corn oil (IV about 118-128), linseed oil (IV about 170-200), rapeseed oil (IV about 100-115), safflower oil (IV about 140-150), sunflower oil (IV about 125-140), tall oil (IV about 140-190), and tung oil (IV about 180) (and mixtures and derivatives thereof) all of which have an adequate number of unsaturated fatty acids (e.g., oleic, linolenic, linoleic) which are suitable for epoxidation.

Typically the unsaturated fatty acids useful in the invention are selected from the random mix of unsaturated fatty acids present in the vegetable oil, the saturated fatty acids are likewise selected from the random mix of saturated fatty acids present in the vegetable oil. The identifying portions of saturated fatty acids present are termed saturated acyl groups that are derived from saturated fatty acids and are typified by palmitoyl, stearoyl, arachidoyl, behenoyl, myristoyl, and margaroyl.

Typical and preferred embodiments of the present invention include the following soybean oil-derived plasticizers which are useful as primary plasticizers in plastics, such as polyvinyl chloride: (i) epoxidized pentaerythritol tetrasoyate; (ii) epoxidized propylene glycol disoyate; (iii) epoxidized ethylene glycol disoyate; (iv) epoxidized methyl soyate; (v) epoxidized sucrose octasoyate; and (vi) the epoxidized product of soybean oil interesterified with linseed oil (epoxidized interesterified soybean oil).

Typically other embodiments of the present invention include hydrolysis products of the equivalent vegetable oil-derived plasticizers, equivalent to those listed immediately above, that are derived from vegetable oils having about the same or higher amounts of unsaturated double bonds. These equivalent vegetable oils include oils having iodine values above 100. Typically oils can vary greatly in iodine number, even within the same type of vegetable oil depending on the growing site of the oil. Typically oils harvested from plants grown in cooler sites will have more double bonds and thus higher iodine values than those grown in warmer regions such as the tropics or subtropics. Accordingly vegetable oils having iodine values above 100 and/or chosen from the group of vegetable oils listed herein are useful in the invention.

For purposes of comparison, derivatives of vegetable oils such as palm oil (IV about 50-55), coconut oil (IV about 7-12), as well as any other varieties of vegetable oil where the IV is below 100, are outside the scope of the invention.

Epoxidized Alkyl Soyates

Any epoxidized alkyl soyate is a particularly suitable candidate for use in the present invention. It is understood that “soyate” is a carboxylate moiety which refers to any naturally occurring or subsequently refined mixture of fatty acids and their esters, where the fatty acids include stearic acid, oleic acid, linoleic acid, linolenic acid, and the like. Epoxidation of such fatty acid esters, such as methyl soyate, typically generates an epoxy group, also called a glycidyl group or oxirane ring, replacing a double bond in the fatty acid backbone.

The epoxidized alkyl soyate can be commercially purchased. Non-limiting examples of epoxidized alkyl soyates include epoxidized methyl soyate, epoxidized ethyl soyate, epoxidized butyl soyate, epoxidized octyl soyate, and combinations thereof. Of these epoxidized methyl soyate (CAS No. 68082-35-9) is preferred.

A commercial source of epoxidized soyates is the Vikoflex® 7010 brand epoxidized methyl soyate from Arkema of Philadelphia, Pa., the reFlex™ 100 brand plasticizer containing epoxidized methyl soyate, the reFlex™ 300 brand plasticizer containing epoxidized methyl soyate (both reFlex™ plasticizers from PolyOne Corporation of Avon Lake, Ohio), and the NEXO® E1 brand epoxidized methyl soyate from Nexoleum of Sao Paolo, Brazil.

Epoxidized alkyl soyate, to be useful in the present invention, should have from about 0.5 to about 12, and preferably from about 5 to about 10 percentage epoxy groups in the molecule.

Production of Epoxidized Alkyl Soyates

If the epoxidized alkyl soyate is to be made rather than purchased, then the epoxidized alkyl soyate can be a reaction product of epoxidized soybean oil (ESO) with an alcohol such as methanol or other alkyl alcohol in the presence of a metallic hydroxide as a catalyst at a temperature of between 23° C.-45° C. and a 1 atmosphere (ambient) pressure and 50% relative humidity for approximately 36 hours using a round bottom flask reaction vessel. ESO, also sometimes called biodiesel, is a known commercial commodity from biological origin.

Another description of the synthesis of epoxidized methyl soyate can be found in Miyagowa et al., “Thermo-Physical and Impact Properties of Epoxy Containing Epoxidized Linseed Oil, 1 Anhydride-Cured Epoxy” Macromol. Mater. Eng. 2004, 289, 629-635.

Other teachings of possible reactions to form epoxidized alkyl soyates and other epoxidized fatty acid esters are found in U.S. Pat. No. 6,797,753 identified above.

Film-Forming Polymers

Generally speaking, film-forming materials such as nitrocellulose, combined, if necessary, with another polymer such as a toluenesulphonamide-formaldehyde resin or an alkyd resin can be used to form films, such as nail varnishes, if combined with a plasticizer to confer good adhesion and good flexibility of the film to the substrate. The plasticizer contributes to the flexibility of the film without weakening its physical strength.

The resin preferably combined with nitrocellulose is the toluenesulphonamide-urea-formaldehyde resin better known under the trade names “Santolite MHP”, “Santolite MS 80%” and “Ketjenflex MS80”. This resin possesses excellent film-forming properties. More preferably, the resin used is an alkyd resin, for example, of the glycerophthalic alkyd type (BECKOSOL ODE). Alkyd resins have the advantages of not containing formaldehyde and of being hypoallergenic.

Nitrocellulose

Nitrocellulose, having CAS No. 9004-70-0, is often used in the application of a film-forming coating to a substrate, with the nitrocellulose being the matrix in which other ingredients reside. Nitrocellulose and those other ingredients are often delivered in the form of a lacquer of nitrocellulose dissolved or substantially dispersed in a volatile organic chemical solvent.

Acceptable solvents are those traditionally used in cosmetic compositions. Among these solvents, one can use ketones such as acetone, methyl ethyl ketone, methyl isobutyl ketone; glycol ethers; alcohols such as ethanol, n-butanol, n-propanol, isopropanol; acetates such as butyl, ethyl and isopropyl acetate, 2-methoxyethyl acetate; linear or branched hydrocarbons such as hexane or octane; or alternatively, aromatic hydrocarbons such as xylene and toluene. Preferably, butyl and ethyl acetates are used.

Nitrocellulose has the monomer repeating unit shown below.

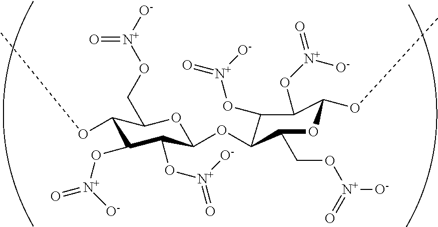

The type of nitrocellulose used in the practice of this invention is not critical except that it must be a soluble grade of nitrocellulose. Soluble nitrocelluloses are those having a nitrogen content from about 10 to about 13% and which are relatively soluble in alcohol.

Nitrocellulose is a polymer consisting of a group of fi-D-glucopyranosyl units substituted to varying degrees with nitrate ester groups. As is the case with all polymers, nitrocellulose does not consist of homogeneous molecules but is a mixture of molecules substituted to a varying extent and having varying molecular weights. Each individual molecule is non-uniform in commercial nitrocellulose grades in that the individual glucopyranose units have from 0 to 3 nitrate ester groups which average 1.7 to 2.6 per glucopyranose unit. The molecular weight of the nitrocellulose can vary between wide limits; the preferred ranges are between a degree of polymerization of 100 monomer repeating units to more than 1500 monomer repeating units. Generally, soluble nitrocelluloses are between 100 monomer repeating units and preferably 700 monomer repeating units in degree of polymerization in order to provide concentrated solutions which do not have an excessive viscosity.

All industrial grades of nitrocellulose must be sold with a regulated minimum level of phlegmatizer for safety. Phlegmatizers can be generally water, alcohol or plasticizer. Occasionally, another solvent is used instead of, or in combination with, one of the others listed. For the purpose of this invention, any of the phlegmatizers which are suitable but the most preferred would be an alcohol and the most common generally (and for nail polish in specifically), are isopropanol, ethanol, and butanol. Non-limiting examples of plasticizers include ESO and acetyl tributyl citrate (ATBC).

The nitrogen content of all commercial industrial grades seem to fall in the range of about 10.5-12.3%. Specifically, nail polish/varnish generally uses high nitrogen content industrial grades in the range of 11.7-12.3% nitrogen.

Commercial grades of industrial nitrocellulose tend to have standard viscosities (ISO 14446) between 7 and 34, desirably 20 to 30, and preferably, for nail varnish, of about 23 to 28.

Nitrocellulose, also known as cellulose nitrate or guncotton, is commercially available from Dow Chemical located in Midland, Mich. bearing the WALSRODER™ Nitrocellulose brand and Nobel NC Company Ltd. located in Thailand bearing the Nobel NC brand. Various grades of such commercial products can be selected without undue experimentation by persons having ordinary skill in the art.

Optional Ingredients

Additives to improve processing or performance of the concentrate of the plasticized nitrocellulose can be added according to preferences of those skilled in the art. For example, functional additives for nitrocellulose can include anti-oxidants, anti-stats, scavengers, blowing agents, biocides, exfoliated nanoclays, thickeners, colorants, other plasticizers compatible with the epoxidized fatty acid esters, and the like, and combinations thereof.

Generally, minor amounts of such additives provide improvement of performance to the nitrocellulose during processing with the other ingredients or in performance of the cellulosic article after manufacturing. One skilled in the art without undue experimentation can determine the appropriate concentration. Particularly useful are colorants which remain in the film on the substrate for aesthetic effect.

Colorants can include at least one pigment of an organic or inorganic nature. Among organic pigments, candidates are D & C Red nos. 10, 11, 12 and 13, D & C Red no. 7, D & C Red nos. 5 and 6, D & C Red no. 34, and lakes such as D & C Yellow no. 5 lake and D & C Red no. 2 lake. Guanine, an organic pigment, is also a candidate.

Inorganic pigments are also useful as cosmetic colorants, such as titanium dioxide, bismuth oxychloride, brown iron oxide and red iron oxides.

Other cosmetic additives include thixotropic rheological agents such as montmorillonite clays, thickeners, pH modifiers such as citric acid, UV screening agents, such as benzophenone derivatives and ethyl 2 cyano-3,3-diphenylacrylate, silicones, and fluorinated agents. Preferably, these additives are used at a concentration at most equal to 1% by weight relative to the total weight of the entire compound.

Table 1 shows acceptable, desirable, and preferable ranges of ingredients useful for film-forming, plasticized polymers, all expressed in weight percent (wt. %) of the entire compound. The compound can comprise, consist essentially of, or consist of these ingredients. Any number between the ends of the ranges is also contemplated as an end of a range, such that all possible combinations are contemplated within the possibilities of Table 1 as candidate compounds for use in this invention.

TABLE 1

| Ingredient (Wt. %) | Acceptable | Desirable | Preferred |
|---|---|---|---|
| Nitrocellulose | 5-35 | 7-35 | 10-30 |
| Volatile Solvent | 40-90 | 50-82 | 50-70 |
| Epoxidized fatty acid ester | 0.5-25 | 3-15 | 5-9 |
| Optional other ingredients | 0-20 | 2-15 | 5-10 |

It has been found that use of the ingredients in the acceptable ranges identified can result in a film as a dry extract of the formulation ranging from 20% to 50% by weight, in order to be useful as a cosmetic product, such as a plain or colorful nail varnish. Desirably, the formulations can be configured to result in dry extracts of about 25-40%, and preferably of about 20-35%.

Processing

Mixing in a batch process typically occurs in a Banbury-type internal mixer operating at a temperature high enough to fuse, or flux, the combination of resin and plasticizer. The temperature can range from about 120° C. to about 320° C. and preferably from about 160° C. to about 250° C. The mixing speeds are typically above 1000 rpm in order to mechanically heat the mixture above the fusion, or flux, point. The output from the mixer is a solid compound in chips or pellets for later dissolution or substantial dispersion into a volatile organic chemical as a safe solvent for use with human fingernails and toenails.

Alternatively, a suitable solvent can be used to dissolve the nitrocellulose at room temperature, followed by adding the epoxidized fatty acid ester and other desired ingredients, followed by storage in suitable containers until application of the liquid mixture onto a surface, and then removal of the volatile solvent to yield a plasticized film.

### USEFULNESS OF THE INVENTION

Film-forming cosmetic composition, for example, nail varnish also called nail polish, whether colorless or colored benefit from use of the epoxidized fatty acid ester as a plasticizer.

Film-forming cosmetic compositions should exhibit the absence of irritation of the substrate, whether skin, nails, or hair, the ease of application, the establishment of a homogeneous film having excellent gloss; a rapid drying time of solvent from matrix forming the film. Nonetheless, the plasticizer needs to assist in providing good adhesion to the substrate and some degree of flexibility. Overall, the film adhered to the nail, the skin (such as an eyelid in the form of mascara), or the hair needs to have sufficient strength to minimize cracks and chipping of the film, particularly if the film also contains vibrant colors.

While cosmetic products have been previously mentioned as useful results of the polymer mixture disclosed above, within reason, almost any product currently made from a solvated nitrocellulose can now be made more bio-derived with improved plasticization performance using an epoxidized fatty acid ester as a plasticizer for that nitrocellulose and other ingredients.

Non-limited examples of uses of nitrocellulose plasticized with epoxidized alkyl soyate include coatings for appliances, such as refrigerators, freezers, washers, dryers, toasters, blenders, vacuum cleaners, coffee makers, and mixers; building and construction, such as fences, decks and rails, floors, floor covering, pipes and fittings, siding, trim, windows, doors, molding, and wall covering; consumer products, such as hand tools, power hand tools, rakes, shovels, lawn mowers, shoes, boots, golf clubs, fishing poles, watercraft, and nail polish; electrical/electronic, such as printers, printing inks, computers, optical films, business equipment, LCD projectors, mobile phones, connectors, chip trays, circuit breakers, and plugs; pharmaceuticals; industrial, such as containers, bottles, drums, material handling, gears, bearings, gaskets and seals, valves, wind turbines, and safety equipment; packaging, such as food and beverage, cosmetic, detergents and cleaners, personal care, pharmaceutical and wellness; transportation, such as automotive aftermarket parts, bumpers, window seals, instrument panels, consoles, under hood electrical, and engine covers; and wire and cable insulating or jacketing for cars and trucks, airplanes, aerospace, construction, military, telecommunication, utility power, alternative energy, and electronics.

An especially desirable use is films, tapes, and sheets, particularly those with pressure-sensitive adhesive on one surface. Currently, many commercially available adhesive tapes made of cellulosic resins rely upon citrate plasticizers.

One reason for such versatility is that the plasticizer is fully bio-derived and the nitrocellulose resin can also be fully bio-derived. In the current marketplace, substantially fully bio-derived polymer compounds are highly desired if their processing, performance, and cost are within currently acceptable ranges.

Preferred epoxidized alkyl soyate-plasticized nitrocellulose can be considered as a plastisol when flowable and the correct powder form of the nitrocellulose is used. A nitrocellulose plastisol can be formed into desired shapes using a variety of coating techniques, as known to those persons having ordinary skill in the art.

Dip Coating: When the plastisol coating becomes a functional part of the mold itself, the process is called dip coating. The metal insert may or may not have a requirement for an adhesive primer. Common uses include tool handles and grips; textiles; wire grates and baskets; plating racks; conveyor hooks; and the like. Dip coating can be either hot dipping or cold dipping.

Hot Dipping: By far the most common dip-coating processing technique, hot dipping requires an item to be heated first before immersion into the plastisol. The heat causes the plastisol coating to gel on the hot form.

Cold Dipping: Preheating the metal part is not required; the amount of pickup obtained depends largely on the viscosity and thixotropic ration of the plastisol.

Molding: Several types of molding are common to plastisol applications. Slush Molding is used to produce hollow, flexible items by filling a mold with plastisol, heating sufficiently to gel a layer next to the inner mold surface, and then draining the excess plastisol. The gelled layer is then completely fused and stripped from the mold. Rotational Molding involves hollow flexible or rigid forms with complex shapes. The process is done using a two-part mold filled with a predetermined weight of plastisol, inserted into a heated oven and rotated on two planes simultaneously. Dip Molding refers to the process of dipping a solid mold; gelling, fusing and stripping the hollow part. Open Molding is a process of molding directly in, or into, a finished article such as automotive air filters.

Other Coating and Casting: Several types of coating employ movement of the plastisol relative to the item or the item relative to the plastisol. Several types of casting employ flow of plastisol on to a stationary substrate. One skilled in the art readily can employ knife coating, roll coating, reverse roll coating, spin coating, solvent casting, etc. according to techniques taught in encyclopedias, other technical literature, or the patent literature, without undue experimentation.

The invention is not limited to the above embodiments. The claims follow.

1. A composite, comprising:
(a) nitrocellulose dissolved in a solvent, and
(b) an epoxidized fatty acid ester plasticizing the nitrocellulose in an amount sufficiently to reduce flexural modulus of the nitrocellulose when solvent is removed and a film of nitrocellulose is formed on a substrate.

2. The composite of claim 1, wherein the composite is a nail varnish, wherein the nitrocellulose has CAS No. of 9004-70-0, wherein the solvent is a ketone, an alcohol, an acetate, a linear or branched aliphatic hydrocarbon, an aromatic hydrocarbon, or combinations thereof, wherein the film is a coating of nail varnish on a human fingernail or toenail, and where the substrate is a human fingernail or toenail.

3. The composite of claim 1, wherein the epoxidized alkyl soyate is selected from the group consisting of epoxidized methyl soyate, epoxidized ethyl soyate, epoxidized butyl soyate, epoxidized octyl soyate, and combinations thereof.

4. The composite of claim 3, wherein the epoxidized alkyl soyate has from about 0.5 to about 12 epoxy groups per molecule.

5. The composite of claim 4, wherein the epoxidized alkyl soyate is the reaction product of epoxidized soybean oil with an alcohol.

6. The composite of claim 3, wherein the nitrocellulose has a nitrogen content of from about 10 to about 13%.

7. The composite of claim 1, further comprising a functional additives selected from the group consisting of anti-oxidants, anti-stats, scavengers, blowing agents, biocides, exfoliated nanoclays, thickeners, colorants, and the like, and combinations thereof.

8. The composite of claim 7, wherein the composite has the following ingredients in the following approximate ranges:
Nitrocellulose 5-35 weight percent
Volatile Solvent 40-90 weight percent
Epoxidized fatty acid ester 0.5-25 weight percent
Optional Functional Additives 0-20 weight percent.

9. The composite of claim 1, wherein the composite has a shape of a coating on a sheet, a tape, a molded article, a coated article, a dipped article, an extruded article, a solvent cast article, or a calendered article.

10. A method of using an epoxidized alkyl soyate as a plasticizer for a nitrocellulose to make a composite of claim 1, comprising the step of mixing the soyate with the nitrocellulose at a temperature sufficient to form a plasticized composite.

11. The method of claim 10, wherein the temperature is at ambient.

12. The method of claim 10, wherein the temperature is above ambient.

13. The method of claim 10, wherein, before the mixing step occurs, the nitrocellulose is dissolved in a solvent and wherein, after the mixing step occurs, the solvent is evaporated.

14. The method of claim 13, wherein the solvent comprises a ketone.

15. The method of claim 12, wherein the nitrocellulose has a nitrogen content of from about 10 to about 13%.

16. The method of claim 10, wherein after the mixing step, the method comprises coating the composite onto a final article made by molding, extruding, dipping, coating, calendering, or solvent casting.

17. The method of claim 16, wherein the shaping step uses substantially the same equipment as used for the composite.

18. An article made of a composite of claim 1.

19. An article made using the method of claim 10.

20. The article of claim 18, wherein the article is selected from the group consisting of appliances; building and construction products; consumer products; electrical/electronic products; packaging; industrial products; transportation products; and wire and cable insulating or jacketing; films; tapes; sheets; and combinations thereof.
